(19) United States
(12) Patent Application Publication
(10) Pub. No.: US 2010/0081191 A1
(43) Pub. Date: Apr. 1, 2010

(54) ANISOTROPIC HEAT SPREADER FOR USE WITH A THERMOELECTRIC DEVICE

(75) Inventor: Mark C. Woods, Dallas, TX (US)

Correspondence Address: BAKER BOTTS L.L.P., 2001 ROSS AVENUE, SUITE 600, DALLAS, TX, 75201-2980, US

(73) Assignee: Marlow Industries, Inc., Dallas, TX (US)

(21) Appl. No.: 12/567,416

(22) Filed: Sep. 25, 2009

Related U.S. Application Data
(60) Provisional application No. 61/100,569, filed on Sep. 26, 2008.

(51) Int. Cl. C12M 1/00; F25B 21/02

(52) U.S. Cl. 435/303.1; 62/3.2

(57) ABSTRACT

A well block for use with a Polymerase Chain Reaction (PCR) Cycler may include a body for holding a plurality of specimen vials, a base for attaching the well block to a temperature control device, and a temperature plate coupled to the base. Further, the temperature plate may include an anisotropic material for transferring thermal energy between the body and the temperature control device.

### CROSS REFERENCE TO RELATED APPLICATIONS

This application claims the benefit under 35 U.S.C. §119(e) of U.S. Provisional Application No. 61/100,569, entitled “Thermoelectric Device Incorporating an Anisotropic Heat Spreader,” filed Sep. 26, 2008.

### TECHNICAL FIELD

The present disclosure relates generally to thermoelectric devices and more specifically to an anisotropic heat spreader for use with a thermoelectric device.

### BACKGROUND

The basic theory and operation of thermoelectric devices has been developed for many years. Presently available thermoelectric devices used for temperature control applications typically include an array of thermocouples which operate in accordance with the Peltier effect. Such thermoelectric devices may also be used for applications such as power generation and temperature sensing.

Thermoelectric devices may be described as essentially small heat pumps which follow the laws of thermodynamics in the same manner as mechanical heat pumps, refrigerators, or any other apparatus used to transfer heat energy. A principal difference is that thermoelectric devices function with solid state electrical components (thermoelectric elements or thermocouples) as compared to more traditional mechanical/fluid heating and cooling components. The efficiency of a thermoelectric device is generally limited to its associated Carnot cycle efficiency reduced by a factor which is dependent upon the thermoelectric figure of merit (ZT) of the materials used in fabrication of the associated thermoelectric elements. Materials used to fabricate other components such as electrical connections, hot plates, and cold plates may also affect the operational characteristics of the resulting thermoelectric device. Typically, a thermoelectric device incorporates both P-type and N-type semiconductor alloys as the materials in the thermoelectric elements.

### SUMMARY

Various industry applications for thermoelectric devices may place particular importance upon thermal uniformity across the surface of a thermoelectric device. For instance, thermoelectric coolers are now widely used in thermal cyclers for performing Polymerase Chain Reactions (PCR) to replicate DNA samples. During PCR, an automated thermal cycler may use a thermoelectric device to rapidly heat and cool a number of test tubes, each containing a sample reaction mixture (e.g., a DNA sample). The heating and cooling process typically includes three steps—denaturation, annealing, and extension—that are repeated for 30 or 40 cycles. During each cycle, each DNA sample is duplicated or amplified and increases exponentially throughout the process. If the samples are heated or cooled for too long during any one step, some or all of the samples may not replicate properly. Accordingly, it may be important for the cycler to be able to rapidly change temperature.

Unwanted or unexpected temperature variations (e.g., hot spots or cold spots) on the surface of the thermoelectric device may cause uneven heating and/or cooling of the DNA samples during the denaturation, annealing, and extension steps. This may lead to a less than optimal result, such as improper replication of the DNA samples in some of the test tubes. Thus, two important characteristics of a PCR thermal cycler include: (1) thermal uniformity across the specimen contact surface (e.g., the surface used to heat and cool the test tubes), and (2) cycling speed (e.g., the rate at which the specimen contact surface can change temperature). In conventional PCR cycler constructions, those two goals are often at odds with each other because increasing the thermal uniformity of the specimen surface often calls for increasing the thermal mass between the thermoelectric device included in the thermal cycler and the test tubes. That increase in thermal mass may lead to a decrease in cycling speed.

One approach seeks to solve the non-uniformity/cycling speed problem by using a re-circulating liquid metal (e.g., gallium) as the thermal interface between the thermoelectric device and the specimens. This approach may exhibit good thermal uniformity and good cycling speed, but the liquid metal may be difficult to manage and may present containment problems. Thus, another solution employing solid components may be preferable.

In view of the points mentioned above, a well block for use with a Polymerase Chain Reaction (PCR) Cycler may include a body for holding a plurality of specimen vials, a base for attaching the well block to a temperature control device, and a temperature plate coupled to the base. Further, the temperature plate may include an anisotropic material for transferring thermal energy between the body and the temperature control device.

In particular embodiments, the base of the well block may include a substantially flat surface intended to face the temperature control device, and the temperature plate may include a generally flat plate of the anisotropic material. Further, the body of the well block my overlie the substantially flat surface and the temperature plate. Also, the temperature plate may be configured to conduct thermal energy more efficiently parallel to the plane of the substantially flat surface than perpendicular to the plane of the substantially flat surface.

Depending upon design, the temperature plate may be integrated into the base such that the combination of the base and the temperature plate form the substantially flat surface.

The well block may further include a plurality of thermoelectric elements having first ends coupled to the temperature plate and second ends coupled to a ceramic plate. The plurality of thermoelectric elements may be electrically interconnected with one another and operable to transfer thermal energy to and from the temperature plate.

Further, a dielectric layer may be disposed between the first ends and the temperature plate, and a heat sink coupled to the ceramic plate.

Depending upon design, the thermal mass of the heat sink may be greater than or equal to a thermal mass of the well block.

In particular embodiments, the temperature plate is coupled to the base by solder. Also it may be the case that the temperature plate is recessed into the base, such that the combination of the base and the temperature plate form the substantially flat surface.

Depending upon design, the base and the body may include a thermally conductive material other than the anisotropic material, and the coefficient of thermal expansion (CTE) of the material may generally be equal to the CTE of the anisotropic material.

Particular embodiments of the well block may also include a plurality of wells overlying the temperature plate, each well defined by an inner surface configured to hold one of the plurality of specimen vials mentioned above.

An anisotropic plate for use in a thermoelectric device may include a plate of anisotropic material. The plate of anisotropic material may have a first substantially flat surface on a first side surrounded by a narrow edge and a dielectric layer on a second side, opposite the first side.

Also, the plate of anisotropic material may be configured to conduct thermal energy more efficiently parallel to the plane of the substantially flat surface than perpendicular to the plane of the substantially flat surface.

Depending upon design, the dielectric layer may include a ceramic plate coupled to the plate of anisotropic material. For example, in particular embodiments, the ceramic plate may include a thin substantially flat sheet of ceramic that is integrated into a second side of the plate of anisotropic material, such that the combination of the ceramic plate and the plate of anisotropic material form a second substantially flat surface on the second side of the anisotropic plate. Also, it may be the case that the first substantially flat surface on the first side of the anisotropic plate may be generally parallel to the second substantially flat surface on the second side of the anisotropic plate.

In particular embodiments, the ceramic plate may be coupled to the plate of anisotropic material by epoxy.

Depending upon design, the dielectric layer may be generally coextensive with the second side of the plate of anisotropic material.

The anisotropic plate may also include a plurality of thermoelectric elements having first ends coupled to the dielectric layer and second ends coupled to a second plate. The plurality of thermoelectric elements may be electrically interconnected with one another via a plurality of electrical interconnects and may be operable to transfer thermal energy to and from the plate of anisotropic material through the dielectric layer. Depending upon design, it may be the case that the second plate includes ceramic. It may also be the case that the second plate includes the anisotropic material.

### BRIEF DESCRIPTION OF THE DRAWINGS

For a more complete understanding of the present disclosure and its advantages, reference is now made to the following descriptions, taken in conjunction with the accompanying drawings, in which:

FIG. 1 illustrates an example embodiment of a thermal cycler that may be used for performing Polymerase Chain Reactions (PCRs) according to an example embodiment of the present disclosure;

FIGS. 2A and 2B illustrate isometric views of an example embodiment of a well block that may be used in the thermal cycler of FIG. 1;

FIG. 3 illustrates an example embodiment of an anisotropic plate that may be used in the thermal cycler of FIG. 1;

FIGS. 4A-4C illustrate example steps in a process that may be used to fabricate the anisotropic plate of FIG. 3; and

FIG. 5 illustrates an example embodiment of a thermal cycler that may be used for performing Polymerase Chain Reactions (PCRs) according to another example embodiment of the present disclosure.

### DETAILED DESCRIPTION

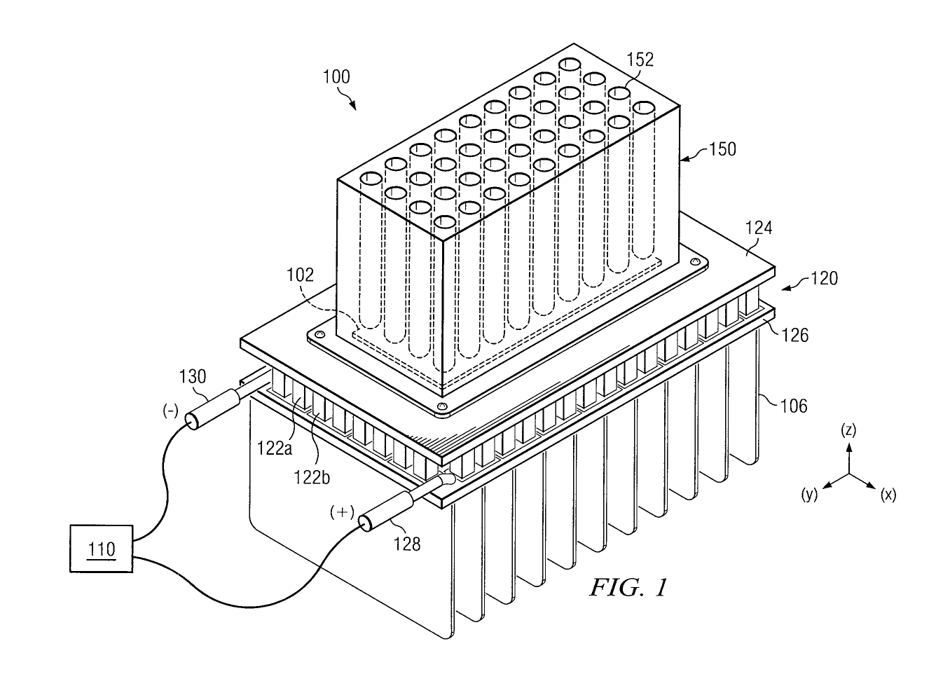

FIG. 1 illustrates an example embodiment of a PCR cycler (“cycler 100”) that may be used for PCR applications. Depending upon design, cycler 100 may include a well block 150 having a plurality of wells 152 for holding a plurality of sample containers (e.g., test tubes), a thermoelectric device 120 for heating and cooling well block 150, a temperature plate 102 for equalizing temperature non-uniformities between well block 150 and thermoelectric device 120, and a heat sink 106 for discharging thermal energy from or supplying thermal energy to thermoelectric device 120. In particular embodiments, cycler 100 may also include a temperature plate 102 coupled between thermoelectric device 120 and heat sink 106 for equalizing temperature non-uniformities between heat sink 106 and thermoelectric device 120. For reference purposes, various components of cycler 100 may be referred to as having a top side intended to face away from thermoelectric device 120 and a bottom surface intended to face toward thermoelectric device 120 (e.g., to be placed upon thermoelectric device 120). Though particular features of those components may be explained using such intended placement as a point of reference, this method of explanation is not meant to limit the scope of the present disclosure to any particular configuration or orientation of those components.

The operation of cycler 100 may be controlled by a control circuit 110. Control circuit 110 may be any component of hardware and/or software capable of electronically controlling the thermal action (e.g., the heating and cooling) of thermoelectric device 120. As an example and not by way of limitation, control circuit 110 may be a microprocessor encoded with logic for controlling the current supplied to thermoelectric device 120. In the context of PCR applications, control circuit 110 may cause thermoelectric device 120 to heat and cool well block 150 in accordance with the steps of a PCR. In various embodiments, control circuit 110 may be hardwired into cycler 100 or integrated into some other component of cycler 100 such as thermoelectric device 120. Alternatively, control circuit 110 may be remote from cycler 100 and connected to thermoelectric device 120 via wires or any other suitable form of connection. In either case, once control circuit 110 is activated, it may be used to control the temperature cycles of thermoelectric device 120.

Operating under the control of control circuit 110, thermoelectric device 120 may heat and cool well block 150 as needed to perform PCR replication. Typically, thermoelectric device 120 includes a plurality of thermoelectric elements 122 (sometimes referred to as “thermocouples”) disposed between a first plate 124 and a second plate 126. Electrical connections 128 and 130 may be provided to allow thermoelectric device 120 to be electrically coupled with an appropriate source of electrical power which may be regulated by control circuit 110.

Thermoelectric elements 122 typically include a plurality of P-type elements 122a and N-type elements 122b arranged in an alternating pattern. That is, P-type elements 122a may be alternating arranged with N-type elements 122b with a dielectric barrier (e.g., an air gap) separating each adjacent P-type element 122a and N-type element 122b. Typically, P-type elements 122a and N-type elements 122b are fabricated from semiconductor materials with dissimilar characteristics and may be connected with one another electrically in series and thermally in parallel. This arrangement enables elements 122 to cooperatively heat one side of thermoelectric device 120 and cool the other. Depending upon the polarity of the current supplied to elements 122, either side of thermoelectric device 120 may be heated or cooled by thermoelectric elements 122. The phrase “semiconductor materials” is used in this application to include semiconductor compounds, semiconductor alloys and mixtures of semiconductor compounds and alloys exhibiting thermoelectric properties.

Ceramic materials are frequently used to manufacture plates 124 and 126. However, plates 124 and 124 may also be made from any other material suitable for use as a substrate for elements 122. As an example and not by way of limitation, plates 124 and 126 may be fabricated from a flexible material such as a strip of polyimide tape or a sheet of copper or other metallic substance coated with a dielectric film. In particular embodiments, if the plates of thermoelectric device 120 (e.g., plates 124 and 126) are rigid, they may be diced part of the way, or completely, through to promote plate flexibility during heating and cooling of thermoelectric device 120. This dicing may appear as a grid-like series of channels cut into plates 124 and/or 126.

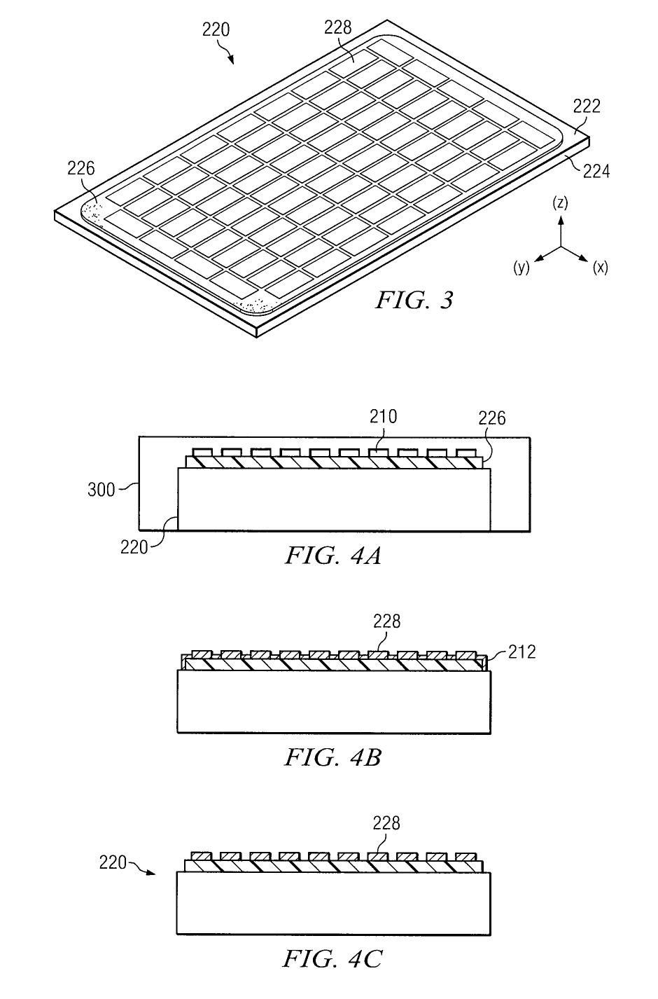

Thermoelectric elements 122 may be electrically connected to one another by a patterned metallization (e.g., circuitry), similar or identical to the electrical interconnects 228 illustrated in FIG. 3, formed on the inward facing sides of plates 124 and 126. Consequently, if an electrically conductive material is used for plates 124 and/or 126, a dielectric barrier may need to be deposited between the patterned metallization (e.g., circuitry) and the electrically conductive portion of the plate to keep thermoelectric elements 122 from short circuiting. If plates 124 and/or 126 are diced all of the way through, care should be taken to cut between the electrical interconnects so as not to damage the circuitry on the inward facing side of the plate(s). One of ordinary skill in the art will appreciate that the above-described embodiments of thermoelectric device 120 were presented for the sake of explanatory simplicity and will further appreciate that the present disclosure contemplates any suitable thermoelectric device 120 constructed from any suitable configuration of components mentioned herein.

Heat sink 106 may be any component or fixture configured for attachment to thermal electric device 120 capable of serving as a reservoir for thermal energy. Once thermoelectric device 120 is coupled between heat sink 106 and well block 150, thermoelectric device 120 may transfer thermal energy from well block 150 to heat sink 106 or vice versa. For example, if thermoelectric device 120 is in the process of cooling well block 150, thermoelectric device 120 may transfer thermal energy out of well block 150 and into heat sink 106, in which case heat sink 106 acts as a thermal reservoir that absorbs thermal energy. Conversely, if thermoelectric device 120 is in the process of heating well block 150, thermoelectric device 120 may draw residual thermal energy out of heat sink 106 and transfer it to well block 150. To help ensure that heat sink 106 is able to absorb or supply an approximately equal amount of thermal energy as well block 150, heat sink 106 may have a thermal mass that is greater than or approximately equal to well block 150.

Typically, heat sink 106 is a passive element, such as for example, a generally solid block of thermally conductive material (e.g., metal) that may be coupled to the opposite side of thermoelectric device 120 from well block 150. Though heat sink 106 may be configured in any desired shape, particular embodiments of heat sink 106 may be configured as a fin structure comprising a series of channels and fins through which air may flow. This configuration may be especially useful when heat sink 106 is used in cooling applications.

As mentioned above, in particular embodiments, a temperature plate 102 may be coupled between heat sink 106 and thermoelectric device 120. This may be done using similar or identical techniques to those discussed below with respect to coupling a temperature plate 102 between well block 150 and thermoelectric device 120. As an example and not by way of limitation, temperature plate 102 may be integrated into the bottom side of heat sink 106 and the combination of those two components soldered to plate 126 of thermoelectric device 120.

Depending upon construction, thermoelectric device 120 may exhibit temperature non-uniformities (e.g., hot spots and cold spots) on plates 124 and 126 during heating and cooling processes. As an example and not by way of limitation, a typical thermoelectric device 120 may exhibit an initial five degrees Celsius (5° C.) to ten degrees Celsius (10° C.) variation in external surface temperature across either of plates 124 and 126. For example, during a heating cycle, plate 124 may be ten degrees warmer at its center than at its edges. Those non-uniformities may be present for any number of reasons, such as for example, variation or imperfections in thermoelectric elements 122, variation or imperfections in the solder joints and or circuitry connecting thermoelectric elements 122, non-uniform heat sinking from heat sink 106, and/or heat transfer from the edges of thermoelectric device 120 into the surrounding ambient air (“edge heat losses”). This variation may increase over the life of the thermoelectric device 120, leading to a markedly irregular surface temperature across thermoelectric device 120 during operation.

As mentioned above, when heating or cooling well block 150 in accordance with the steps of a PCR, it may be desirable to maintain thermal uniformity across the bottom side of well block 150 (e.g., the side facing thermoelectric device 120) so that each of the specimens will be subjected to uniform temperature conditions. For instance, during the denaturation step, if the temperature of a particular specimen is too cold, the DNA sample contained therein may not melt and open into the single-stranded DNA. By contrast, if the temperature of a particular specimen is too hot, the enzymes mixed with the DNA sample contained therein may overheat and char. In either case, the desired reaction may not take place for that specimen.

The degree to which the non-uniform temperature field of thermoelectric device 120 is reflected in well block 150 may be affected by the thermal interface between thermoelectric device 120 and well block 150. To help minimize that non-uniformity, a temperature plate 102 may be inserted between thermoelectric device 120 and well block 150 to more evenly distribute the thermal energy from thermoelectric device 120 across the bottom side of well block 150. In other words, it may be the job of temperature plate 102 to even out any temperature non-uniformities present in thermoelectric device 120 as it conducts the thermal energy from thermoelectric device 120 to well block 150. Depending upon design temperature, temperature plate 102 may be coupled to, or integrated into, well block 150 as discussed below. In an alternative embodiment, a specimen slide containing one or more specimens may be clamped against temperature plate 102 in place of well block 150.

Temperature plate 102 may be any component or fixture of material that is generally capable of distributing thermal energy across the base 154 of well block 150. Furthermore, temperature plate 102 may be coupled to well block 150 using any suitable mechanism or method. As an example and not by way of limitation, temperature plate 102 may be an independent plate of material that may be mechanically coupled to the bottom side of well block 150 using screws or bolts. As another example and not by way of limitation, temperature plate 102 may be a plate of material that has been integrated into, or soldered to, the bottom side of well block 150 such that it may not be readily removed. In any case, once coupled to the bottom side of well block 150, temperature plate 102 may act as a heat spreader to evenly distribute thermal energy from thermoelectric device 120 across the bottom side of well block 150.

When placed upon thermoelectric device 120, temperature plate 102 may be coupled thereto using any suitable mechanism or method. For example, in particular embodiments, temperature plate 102 may be mechanically coupled to thermoelectric device 120 using for example bolts or screws. In this scenario, an interface material (e.g., a grease) may be placed under compression between thermoelectric device 120 and temperature plate 102 to enhance the thermal interface between those components. In other embodiments, temperature plate 102 may be soldered or epoxy-bonded directly to one of the plates of thermoelectric device 120.

As the thermal interface between thermoelectric device 120 and temperature plate 102 improves, any non-uniformities in the surface temperature of thermoelectric device 120 may be increasingly exhibited on the bottom side of the temperature plate 102 (e.g., the side facing thermoelectric device 120). One way to increase temperature plate 102's ability to even out those non-uniformities is to increase its thickness. However, as briefly mentioned above, this solution may sacrifice the cycling speed of cycler 100 since cycling speed is inversely proportional to the thermal mass of temperature plate 102. That is, the larger the thermal mass residing between thermoelectric device 120 and the specimen vials, the longer it may take for thermoelectric device 120 to heat and cool those vials.

Other solutions for reducing thermal non-uniformities aside from increasing the thermal mass between thermoelectric device 120 and well block 150 include judicious well block design, spacing of multiple modules, control system improvement, and the inclusion of heaters to minimize edge effects. However, those solutions may be expensive, complex, and difficult to implement.

Yet another solution for reducing thermal non-uniformities exhibited by thermoelectric device 120 that may overcome the above-mentioned drawbacks, is to manufacture temperature plate 102 out of a material having anisotropic heat transfer characteristics. As opposed to isotropic materials which conduct thermal energy with generally equal efficiency in all directions, anisotropic materials have the ability to conduct heat more efficiently in one direction than in another. For convention herein, the term “anisotropic” will be used to describe materials that conduct thermal energy at least three times more efficiently in one direction than in another. The term “isotropic” material will be used to refer to materials having directional thermal conductivities falling below that ratio.

As an example, in the pictured embodiment, temperature plate 102 may be fabricated from an anisotropic material that is five times more efficient at conducting thermal energy laterally across temperature plate 102 (e.g., along the X-axis and Y-axis) that through the width of temperature plate 102 (e.g., along the Z-axis). Consequently, when heated, such materials may promote thermal uniformity across the surface of temperature plate 102 (e.g., in the X-Y plane) while still exhibiting good through-plane heat conduction (e.g., along the Z-axis).

By constructing temperature plate 102 out of an anisotropic material, the amount of thermal mass needed for temperature plate 102 to effectively spread heat may be reduced as compared to temperature plates constructed out of isotropic materials. This may enable temperature plate 102 to efficiently smooth out any non-uniformities of thermoelectric device 120's temperature field with minimum thermal mass penalty. Thus, this solution may provide high thermal uniformity across temperature plate 102 with minimum penalty in cycling speed.

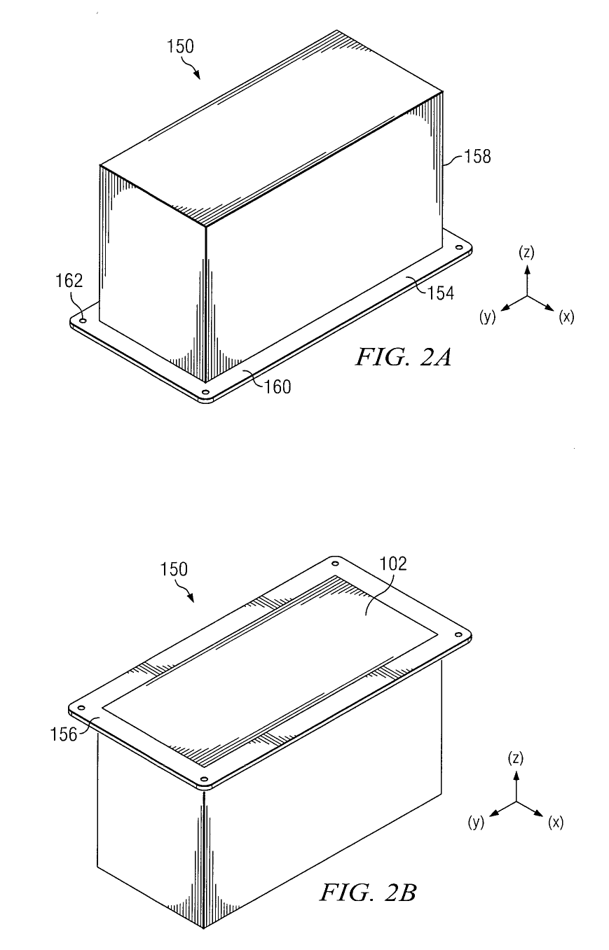

FIGS. 2A and 2B illustrate isometric views of an example embodiment of well block 150 in isolation from the other components of cycler 100. In particular, FIG. 2A illustrates an isometric view of the topside of well block 150 before wells 152 have been created, and FIG. 2B illustrates an isometric view of the bottom side of well block 150. In both views, well block includes a base 154 having a generally flat surface 156 lying in the X-Y plane and a body 156 extending out of base 154 along the Z-axis.

As illustrated in FIG. 2B, temperature plate 102 may be integrated into base 154 on the bottom side of well block 150. Base 154 may be any extension, component, or fixture on well block 150, or combination thereof, capable of being used to attach well block 150 to thermoelectric device 120. As one example, base 154 may be a solderable surface located on the bottom side of well block 150. As another example and not by way of limitation, base 154 may be one or more lateral extensions 160 extending laterally from body 158 that may include one or more attachment points 162 for attaching well block 150 to thermoelectric device 120. An attachment point 162 may be any mechanism or fixture operable to serve as a rigid point of attachment between lateral extension 160 and thermoelectric device 120. As one example and not by way of limitation, an attachment point 162 may be a screw hole configured to accept a screw or a bolt. As another example an not by way of limitation, an attachment point 162 may be a solder bump deposited on the under side of lateral extension 160. One of ordinary skill in the art will appreciate that the above-described embodiments of base 154 and attachment points 162 were presented for the sake of explanatory simplicity and will further appreciate that the present disclosure contemplates the use of any suitable type of base 154 including any suitable number and type of attachment points 162 for attaching well block 150 to thermoelectric device 120.

Body 158 may be any extension, component, or fixture on well block 150 capable of accommodating a plurality of wells 152 for holding a plurality of specimen vials (e.g., test tubes). As an example and not by way of limitation, body 158 may comprise a plurality of interconnected vertical wells 152 extending out of base 154 generally parallel to the Z-axis. Each well 152 may be defined by an inner surface of body 158 surrounding a generally concave opening, such as for example, a cone-shaped or cylindrically-shaped opening configured to hold a specimen vial. Wells 152 may be created, for example, by drilling holes into body 158.

Typically, base 154 and body 158 are integrally connected and are fabricated the same piece of material. Although any suitable material or combination of materials may be used, in general, base 154 and body 158 are fabricated from a single piece of rigid, thermally conductive material such as metal.

As mentioned above, in particular embodiments, temperature plate 102 may be integrated into base 154. For example, temperature plate 102 may be soldered or epoxied into a recession that has been carved into the bottom side of base 154 such that the combination of base 154 and temperature plate 102 form generally flat surface 156. As another example, temperature plate may be forged into base 154 using a process similar to that described below with respect to FIG. 4 (e.g., temperature plate 102 may be placed into a mold along with well block 150, and those two components may be encapsulated together by a thin metal shell formed by injecting a molten metal alloy around them in the mold). Depending upon design, temperature plate 102 may cover any portion of generally flat surface 156 and may be any thickness. For example, in one embodiment, temperature plate 102 may be approximately one millimeter (1 mm) thick and may cover the entirety of generally flat surface 156.

Temperature plate 102 may be fabricated from any suitable anisotropic material. As an example and not by way of limitation, temperature plate 102 may be fabricated from a laminate of thermal pyrolytic graphite (“TPG”) wherein the carbon fibers in the TPG are aligned along the X-Y plane so as to conduct heat more effectively in plane (e.g., within the X-Y plane) than through plane (e.g., along the Z-axis). In another example embodiment, temperature plate 102 may be composed of an orthotropic aluminum graphite flake composite developed by Metal Matrix Cast Composites, Inc., sold under the trade name AlGrp™ Particular embodiments of this material may have an in-plane thermal conductivity (k_{xy}) of ˜700 W/m-K and a through-plane thermal conductivity (k_{z}) of ˜40 W/m-K. One of ordinary skill in the art will appreciate that the above-described examples of anisotropic materials were presented for the sake of explanatory simplicity and will further appreciate that temperature plate 102 may be composed of any suitable anisotropic material.

To help prevent temperature plate 102 and/or well block 150 from cracking due to thermal expansion, the anisotropic material used for temperature plate 102 may have a low in-plane (e.g., in the X-Y plane) coefficient of thermal expansion (CTE) which may be tuned to match the CTE of the material of well block 150 by adjusting the recipe of the anisotropic material.

This same process may also be used to match the CTE of the material used for temperature plate 102 with the CTE of the material used for one of the plates of thermoelectric device 120 (e.g., plates 124 and 126). For example, in one embodiment, a manufacturer may tune the CTE of the anisotropic material used for temperature plate 102 to match the CTE of the ceramic used to construct the plates of thermoelectric device 120 (e.g., plates 124 and 126). More particularly, plates 124 and 126 could be manufactured from an alumina ceramic having a CTE ˜7 ppm/° C. and the CTE of the anisotropic material used for temperature plate 102 may be tailored to match. The anisotropic nature of the anisotropic material used for temperature plate 102 may result in highly uniform external surface temperature, while the low CTE may make it possible to directly-attach thermoelectric device 120 to temperature plate 102 without a grease joint.

FIG. 3 illustrates an example anisotropic plate 220 that may be used in place of one or both of plates 124 and 126 in thermoelectric device 120. Though anisotropic plate 220 may created in any desired shape, typically, anisotropic plate 220 includes a generally flat surface 222 surrounded by a narrow edge 224. In the pictured embodiment, generally flat surface 222 lies in the X-Y plane.

Anisotropic plate 220 may be fabricated from any suitable anisotropic material including those mentioned above with respect to temperature plate 102. Typically, anisotropic plate 220 is constructed such that it conducts thermal energy more efficiently parallel to the plane of generally flat surface 222 (e.g., the X-Y plane) than perpendicular to the plane of generally flat surface 222 (e.g., along the Z-axis). If the anisotropic material used in anisotropic plate 220 is electrically conductive, a dielectric layer 226 may be deposited on, or integrated into, anisotropic plate 220 to provide electrical insulation between the anisotropic material and the electrical interconnects 228 that may be used to interconnect thermoelectric elements 122. Depending upon design, dielectric layer 226 may cover any portion of generally flat surface 222 and may be any thickness. For example, in one embodiment, dielectric layer 226 may be a ceramic plate approximately ten millimeters (10 mm) thick covering the entirety of generally flat surface 222.

More generally, dielectric layer 226 may be any deposition or fixture, or combination thereof, coupled to anisotropic plate 220 capable of electrically insulating thermoelectric elements 122 from the anisotropic material in anisotropic plate 220. Further, dielectric layer 226 may be composed of any suitable dielectric substance having a relatively high thermal conductivity (e.g., Beryllium Oxide (“BeO”), Aluminum Oxide (“Al₂O₃”), or Aluminum Nitride (“AlN”)). As an example and not by way of limitation, dielectric layer 226 may be a thin ceramic or glass plate that has been epoxied into a recession in anisotropic plate 220 as part of generally flat surface 222. As another example and not by way of limitation, dielectric layer 226 may be a thin film deposition of dielectric material. As yet another example and not by way of limitation, dielectric layer 226 may be a rigid plate of dielectric material (e.g., ceramic) that has been forged into anisotropic plate using the method described with respect to FIG. 4 below. In this case, dielectric layer 226 may form the entirety of generally flat surface 222. Once dielectric layer 226 has been incorporated into anisotropic plate 220, it may serve as the electrically insulating substrate upon which the circuitry for thermoelectric device 120 may be built.

If anisotropic plate 220 is included in cycler 100, temperature plate 102 may be eliminated from cycler 100 since anisotropic plate 220 may serve to uniformly distribute the temperature field across the surface of thermoelectric device 120. However, anisotropic plate 220 may have other uses aside from cycler 100. For example, anisotropic plate 220 may be used as the reference surface in highly-uniform thermal reference source with “on-demand” response time. Those sources may be used, for example, to calibrate thermal imagers. When used in this application, the outward facing surface of temperature plate 102 (e.g., the surface opposite dielectric layer 226) may be coated with a high emissivity material to increase anisotropic plate 220's ability to uniformly radiate thermal energy. Such materials may be a highly emissive coating including a transition metal oxide such as chromium oxide (Cr₂O₃), cobalt oxide (CoOₓ), ferrous oxide (Fe₂O₃), or nickel oxide (NiO) as the high emissivity agent.

FIGS. 4A-4C illustrate a series of steps that may be used in an example process for making anisotropic plate 220. In particular, FIG. 4A illustrates a first step of the process wherein dielectric layer 226, in this case a ceramic plate, is placed into a mold 200. Afterwards, anisotropic plate 220 may be placed on top of dielectric layer 226 such that the two plates lie in direct contact. Dielectric layer 226 also separates anisotropic plate 220 from a series of air gaps 210 formed in the bottom of mold 200. Once both plates are placed in mold 200, a molten metal alloy such as an Aluminum Silicon alloy is heated to approximately 575 degrees Celsius (575° C.) and then injected into mold 220. This is done under pressure to form a thin (e.g., 0.001 inch-thick) metal layer 212 of the metal alloy around both dielectric layer 226 and anisotropic plate 220 as generally illustrated in FIG. 4B. After the molten metal alloy has cooled, metal layer 212 may completely encapsulate anisotropic plate 220 and dielectric layer 226, holding them together. During injection, the molten metal alloy may also fill air gaps 210 to form electrical interconnects 228 on the face of dielectric layer 226. Next, the portions of metal layer 212 located around electrical interconnects 228 may be removed, for example by spray etching, to electrically isolate electrical interconnects 228 from each other and from anisotropic plate 220 as illustrated in FIG. 4C. Once this has been completed, electrical interconnects 228 may be plated with Nickel and/or Gold to provide a solderable surface for thermoelectric elements 122, after which, the process ends.

In other applications, a heat producing device (e.g., one or more electrical or optical components such as for example a CPU, a GPU, a laser diode, or a laser diode bar) may be coupled to the upper surface of the temperature plate 102 in place of well block 150 to transfer heat from the heat producing device to the tops of thermoelectric elements 122. Furthermore, in power generation applications, one or both of plates 124 and 126 may be replaced by anisotropic plate 220 to effectively spread heat from a point source to thermoelectric elements near the fringes of the thermoelectric device 122.

Other applications for anisotropic materials may include use in an evaporator unit to effect nucleate boiling heat transfer while staying beneath critical heat flux limits, use as a natural and or forced convection heat sink base plate for thermoelectric cooler applications, use as a substrate for thermoelectric devices (e.g., using a dielectric layer between the anisotropic material and the electrical contacts or soldering alumina chips having copper pads using high-temp solder, and then building thermoelectric devices with a lower temperature solder), use as an improved substrate for planar multi-stage thermoelectric coolers to spread heat more effectively between the stages, use as both a thermoelectric device substrate and optical bench heat spreader (e.g., laser/telecom apps), use as a thermoelectric device cold finger or net-shape casting of a cold finger integrated with a wall, use as the fins in a heat sink to which thermo electric devices are mounted, use of a nickel-plated version of the anisotropic material as the base plate for a hermetically sealed package, use in a thermoelectric device using anodized outer aluminum skin, use as a “Collector” for a thermoelectric generator to harvest over a large area, use as a heat spreader for high-watt density thermoelectric devices, use as a well block enabling the thermoelectric circuit to be built directly on the well block (e.g., with an intermediary dielectric layer), and use in tooling for high-temperature solder reflows to assist in uniform cooling during the manufacturing of thermoelectric coolers.

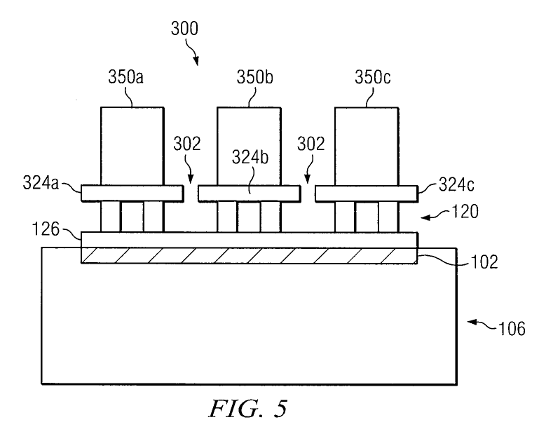

FIG. 5 illustrates an example thermal cycler (“cycler 300”) according to another example embodiment of the present disclosure. Cycler 300 is generally identical to Cycler 100 except that: well block 150 has been replaced by a plurality of discrete well blocks 350a-c, a plurality of cuts 302 have been diced into to plate 124 to create a number of smaller discrete plates 324a-c, and a temperature plate 102 has been coupled between heat sink 106 and thermoelectric device 120. Although only two cuts 302 are illustrated, any suitable number of cuts 302 may be diced into plate 124 in any suitable configuration to create any suitable number of discrete plates 324.

Depending upon design, each discrete plate 324 may be coupled to a corresponding discrete well block 350. Each discrete well block 350 may be virtually identical to well block 150, except for being generally smaller in size. For example, each discrete well blocks 350 may be sized to fit within the confines of a discrete plate 324. In fact, in particular embodiments, well block 150 may be diced into discrete well blocks 350 at the same time plate 124 is diced into discrete plates 324. This may be accomplished, for example, by soldering well block 150 to plate 124 and dicing the combination of those two components into segments, each segment including a discrete well block 350 coupled to a discrete plate 324.

In embodiments where well block 150 and plate 124 are made of dissimilar materials, dicing plate 124 and well block 150 into smaller sections may reduce the mechanical stress imposed on those components due to CTE mismatch. More particularly, since well block 150 and plate 124 may expand and contract at different rates when heated and cooled, separating those components into smaller sections may reduce the mechanical stress imposed on the joint between them, eliminating any need for an intermediary material such as a grease joint to absorb the stress. This technique may be especially beneficial in situations where the CTE of well block 150 is vastly different from the CTE of plate 124, because it may enable those components to be bonded together using a rigid intermediary such as solder or epoxy rather than being bonded together using a non-rigid intermediary such as grease.

As mentioned above, cycler 300 also includes a temperature plate 102 coupled between thermoelectric device 120 and heat sink 106. This may generally equalize any temperature non-uniformities exhibited by thermoelectric device 120 on heat sink 106. Creating a uniform temperature distribution across heat sink 106 may result in a similar temperature distribution being exhibited on well blocks 350. This may be true even in the absence of a second temperature block 102 between well blocks 350 and thermoelectric device 120. Consequently, in embodiments where a temperature block 102 is coupled between heat sink 106 and thermoelectric device 120, it may be possible to omit the temperature block 102 between well blocks 350 and thermoelectric device 120 while still achieving a relatively uniform temperature distribution across well blocks 350. This may apply equally as well in cycler 100.

Although the present disclosure has been described in several embodiments, a myriad of changes, substitutions, and modifications may be suggested to one skilled in the art, and it is intended that the present disclosure encompass such changes, substitutions, and modifications as fall within the scope of the present appended claims. Moreover, none of the methodology described herein should be construed as a limitation on the order of events insofar as one of skill in the art would appreciate that such events could be altered without departing from the scope of the disclosure.

What is claimed is:

1. A well block for use with a Polymerase Chain Reaction (PCR) Cycler, the well block, comprising:
a body for holding a plurality of specimen vials;
a base for attaching the well block to a temperature control device; and
a temperature plate coupled to the base, the temperature plate comprising an anisotropic material for transferring thermal energy between the body and the temperature control device.

2. The well block of claim 1, wherein:
the base comprises a substantially flat surface intended to face the temperature control device;
the temperature plate comprises a generally flat plate of the anisotropic material;
the body overlies the substantially flat surface and the temperature plate; and
the temperature plate is configured to conduct thermal energy more efficiently parallel to the plane of the substantially flat surface than perpendicular to the plane of the substantially flat surface.

3. The well block of claim 2, wherein the temperature plate is integrated into the base such that the combination of the base and the temperature plate form the substantially flat surface.

4. The well block of claim 3, further comprising a plurality of thermoelectric elements having first ends coupled to the temperature plate and second ends coupled to a ceramic plate, the plurality of thermoelectric elements electrically interconnected with one another and operable to transfer thermal energy to and from the temperature plate.

5. The well block of claim 4, further comprising:
a dielectric layer disposed between the first ends and the temperature plate; and
a heat sink coupled to the ceramic plate.

6. The well block of claim 5, wherein a thermal mass of the heat sink is greater than or equal to a thermal mass of the well block.

7. The well block of claim 2, wherein the temperature plate is coupled to the base by solder.

8. The well block of claim 7, wherein the temperature plate is recessed into the base, such that the combination of the base and the temperature plate form the substantially flat surface.

9. The well block of claim 1, wherein the base and the body comprise a thermally conductive material other than the anisotropic material; and
a coefficient of thermal expansion (CTE) of the material is generally equal to a CTE of the anisotropic material.

10. The well block of claim 2, wherein the body comprises a plurality of wells overlying the temperature plate, each well defined by an inner surface configured to hold one of the plurality of specimen vials.

11. An anisotropic plate for use in a thermoelectric device, the plate, comprising:
a plate of anisotropic material that includes a first substantially flat surface on a first side surrounded by a narrow edge; and
a dielectric layer on a second side, opposite the first side.

12. The plate of claim 11, wherein the plate of anisotropic material is configured to conduct thermal energy more efficiently parallel to the plane of the substantially flat surface than perpendicular to the plane of the substantially flat surface.

13. The plate of claim 12, wherein the dielectric layer comprises a ceramic plate coupled to the plate of anisotropic material.

14. The plate of claim 13, wherein the ceramic plate comprises a thin substantially flat sheet of ceramic that is integrated into a second side of the plate of anisotropic material, such that the combination of the ceramic plate and the plate of anisotropic material form a second substantially flat surface on the second side.

15. The plate of claim 14, wherein the first substantially flat surface is generally parallel to the second substantially flat surface.

16. The plate of claim 13, wherein the ceramic plate is coupled to the plate of anisotropic material by epoxy.

17. The plate of claim 12, wherein the dielectric layer is generally coextensive with the second side of the plate of anisotropic material.

18. The plate of claim 12, further comprising a plurality of thermoelectric elements having first ends coupled to the dielectric layer and second ends coupled to a second plate, the plurality of thermoelectric elements electrically interconnected with one another via a plurality of electrical interconnects and operable to transfer thermal energy to and from the plate of anisotropic material through the dielectric layer.

19. The plate of claim 18, wherein the second plate comprises ceramic.

20. The plate of claim 18, wherein the second plate comprises the anisotropic material.
